(19) United States
(12) Patent Application Publication
(10) Pub. No.: US 2008/0318203 A1
(43) Pub. Date: Dec. 25, 2008

(54) Novel Microfluidic System and Method for Capturing Cells

(76) Inventors: Phuong-Lan Tran, Paris (FR); Bernard Bennetau, Camblanes (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/571,808

(22) PCT Filed: Jul. 7, 2005
(86) PCT No.: PCT/FR2005/001753

§ 371 Date: Aug. 29, 2008

(30) Foreign Application Priority Data
Jul. 9, 2004 (FR) 0407722

(51) Int. Cl. A01N 1/02; C12M 1/00; C12Q 1/02; C12N 11/00

(52) U.S. Cl. 435/2; 435/283.1; 435/174; 435/29

(57) ABSTRACT

The invention concerns a microfluidic device and its uses for capturing subset of cells in a biological fluid. Said device comprises a chamber (1) for circulating fluids whereof an inner wall (5) is provided with a covalent immobilization surface in the form of an organized self-assembled silane layer, whereto is fixed a layer of biomolecules for specific identification of the population of cells.

A subject matter of the invention is a novel microfluidic device and its use in capturing subpopulations of cells in a biological fluid.

More particularly, the invention relates to a device comprising a chamber for circulation of fluids, one wall of which is provided with grafting which makes possible the capture of a subpopulation of cells in a biological sample circulated in this chamber. This device makes possible the capture of cells present in a very small amount in this biological sample. Another subject matter of the invention is a process for capturing cells in a biological sample, this process being characterized in that it comprises a stage of passing the biological sample through such a device.

The device of the invention can be used for the purification and characterization of circulating tumor cells from a blood sample of the patient or for the purification and characterization of fetal cells from a blood sample taken from the mother.

Breast cancer is the commonest malignant tumor in women in Europe and the United States. The monitoring of metastatic dissemination is an important factor in the prognosis of the pathology. The purification and molecular characterization of the circulating tumor cells (CTC) represent an important challenge in the monitoring of patients, both as parameter for assessing the micrometastatic disease at an early stage and as phenotypic and genotypic tool at a late stage.

The possibility of purifying and characterizing circulating tumor cells is of major interest in the diagnosis and monitoring of the development of breast cancer but also of other types of cancer, such as prostate, kidney, bladder, liver, colon or lung cancer.

Current procedures for purifying CTC use the CELLection kit, which operates according to the principle of separation of the cells by capturing by magnetic beads to which the specific antiepithelial antibody is grafted (Dynal® RAM IgGI CELLection™ Kit). This method combines several very cumbersome stages of centrifuging. It requires handling for virtually a day and a half. The yields are very low. CTC losses are high, mainly due to their attachment to the magnetic beads. It is for this reason that this technique is difficult to use in routine clinical application.

Devices comprising a laminar flow chamber which makes possible the capture of cells are known in the prior art.

The document WO 03/091730 discloses a device intended for monitoring the migration of leukocytes, this device comprising two chambers connected to one another via a channel. The objective is to study the mechanisms of binding of the leukocytes to the walls of the blood vessels. Compounds which are mediators of the migration of leukocytes composed of endothelial cells can be placed in the channel. Test compounds can also be placed in this device.

The document US 2003/0044766 discloses a process and a device for detecting interaction between two types of cells, one being fixed in a passage subjected to a laminar flow of a dispersion comprising the second type of cell.

This device is intended to study the interactions between populations of leukocytes and endothelial cells.

The document A. Pierres et al., Faraday Discuss., 1998, 111, 321-330, discloses the use of a laminar flow chamber for studying the formation of bonds between spheres covered with streptavidin and the walls of the chamber grafted by biotin.

The document M. R. Wattenberger et al., Biophys. J., vol. 57, April 1990, 765-777, studies in detail the parameters which govern the interaction between a surface covered by a ligand and cells carrying a receptor. Receptors incorporated in liposomes were used as cell model. The tests were carried out in a laminar flow chamber.

The shear force, the strength of the ligand-receptor bond and the density of receptors on the surface are important parameters.

The document WO 00/23802 discloses a diagnostic method which makes it possible to determine the binding capacity of cells in a biological sample which consists in passing this sample over a surface covered with a substrate which has bonded to the cells.

This document is more particularly directed at the diagnosis of platelet abnormalities and thromboses. The shear conditions which are applied are intended to imitate the conditions which these cells experience in vivo in blood vessels.

However, the documents of the prior art relate essentially to devices and processes intended to study interactions between two cell populations: leukocytes and endothelial cells, to study platelet adhesion or cell models (liposome spheres) carrying receptors.

None of these documents relates to the capture and the study of circulating tumor cells or of fetal cells.

The documents of the prior art relate to the study of the behavior of populations of cells which are present at a high concentration in the samples treated and which are covered with receptors present at their surface at a very high density.

The cells with which the present invention is more particularly concerned have the distinguishing feature of being present at a very low concentration in the biological samples studied, in the midst of other populations of cells which greatly outnumber them. The specific receptors of these cells are expressed at a low density at their surface. It is these two distinguishing features which make it particularly difficult to capture them and identify them in a biological sample.

The use of a laminar flow device of the prior art, one wall of which will be conventionally grafted by a ligand for the CTCs, does not make it possible to capture CTCs in a blood sample as the latter are present in an excessively low amount and the antigen-antibody interaction concerned by this capture is of the order of 50 to 150 piconewtons, which is extremely low in comparison with the interactions of the molecules for adhesion between two populations of cells. The invention relates more particularly to the capture of a population of cells, the specific surface markers for which have an interaction with their receptors with a strength ranging from 10 pN to 1 nN, advantageously from 20 pN to 500 pN, preferably from 30 to 300 pN, more specifically from 50 to 150 pN.

The problems which the present invention is targeted at solving are the capture of a minority cell population of CTC type in a biological sample while using a small amount of biological sample and while having a capture efficiency which is as high as possible.

This problem could be solved by virtue of a device comprising a chamber for circulation of fluids, one internal wall of which is provided with specific grafting.

The grafting with which the device of the invention is provided has the distinguishing feature of exhibiting a homogeneous uniform surface to which can be grafted a population of ligands specific for the subpopulation of cells which it is desired to capture. The surface homogeneity favors low-strength ligand-receptor or antigen-antibody interactions which, under other equipment conditions, would not make it possible to carry out this capture.

A chamber for the circulation of fluids comprises a cavity, preferably a cavity of parallelepipedal type, comprising two orifices placed at two ends of the cavity, the fluid being injected via a first orifice and collected via a second orifice.

The dimensions of the chamber for circulation of fluids are advantageously chosen so that it is a laminar flow chamber. It can also be a cavity of a microfluidic device, such as that disclosed in the document U.S. Pat. No. 6,408,878. Advantageously, the internal cavity of the chamber for circulation of fluids has a volume of less than or equal to 30 μl, more preferably still of less than or equal to 12 μl, which makes it possible to treat low-volume biological samples.

Advantageously, the device is equipped with a pump which makes it possible to inject the sample fluid into the chamber for circulation of fluids. More advantageously, the device comprises a circuit for circulation of the fluid in a loop connected to a pump, so that the sample to be treated passes more than once through the chamber for circulation of fluids before being collected.

According to the present invention, at least one wall of the chamber for circulation of fluids is provided with specific grafting. Preferably, it is a wall parallel to the flow which passes through the chamber for circulation of fluids. Preferably, the chamber for circulation of fluids is a parallelepiped and one of its internal faces comprises specific grafting.

The wall of the chamber for circulation of fluids provided with specific grafting is composed of a solid support which can be a glass or silicon slide or any solid metal surface comprising —OH functional groups at the surface and which is placed in the chamber for circulation of fluids.

The surface of the solid support, glass, Si or other slide, is functionalized by a homogeneous self-assembled monomolecular layer of chlorosilane chains.

A protein entity for recognition of the population of target cells, in particular of circulating tumor cells (antibody or protein ligand) is grafted directly or via an appropriate coupling agent to the layer of chlorosilanes. The selectivity of the recognition biomolecule is determining for the capture of the population of target cells. The solid support (slide) prepared is subsequently mounted in the device for circulation of fluids.

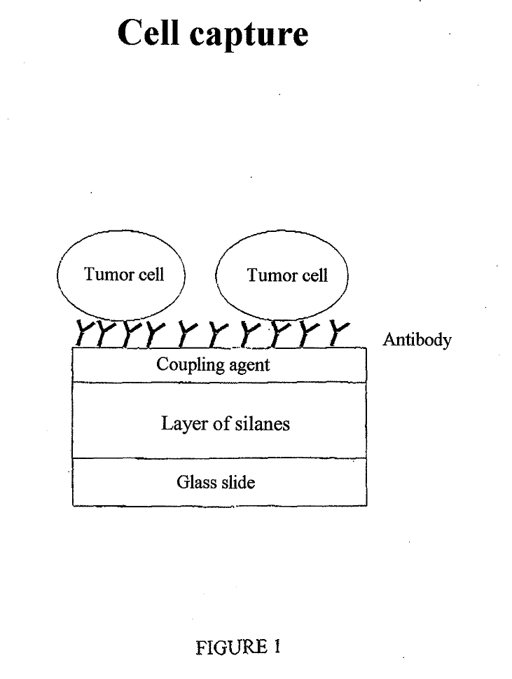

The solid support for cell capture is functionalized in 3 stages: the grafting of chlorosilanes to the glass slide, the grafting of a coupling agent (which can be a chemical coupling agent or an agent of protein A or G type) and, finally, the grafting of a protein recognition entity (antibody or the ligand for a membrane receptor of a cell). FIG. 1 illustrates a glass slide of this type grafted by an antibody: the solid support is grafted by an organized self-assembled layer of silane to which a layer of coupling agent (linker) is fixed via a covalent bond. A layer of biomolecules for specific recognition of the target cell population is covalently or noncovalently fixed to the coupling agent. The presence of the layer of coupling agent is optional: according to one alternative form of the invention, it is possible to provide for the recognition biomolecules to be directly fixed to the organized self-assembled layer of silane.

In order to covalently immobilize organic molecules on an inorganic surface, it is first of all necessary to graft, to this surface, coupling molecules which will ensure the fixing of the organic molecules to the inorganic substrate.

Organosilicon coupling molecules have been provided for this aim by L. A. Chrisey et al. (Nucleic Acids Research, 1996, 24, 15, 3031-3039) and U. Maskos et al. (Nucleic Acids Research, 1992, 20, 7, 1679-1684). The molecules used, namely 3-glycidoxypropyltrimethoxy-silane and various aminosilanes, exhibit, however, the disadvantage of being deposited randomly and non-reproducibly on the surface. They form a nonhomogeneous film thereon, the thickness of which cannot be controlled; in addition, the film is not very robust with regard to subsequent chemical treatments, the non-homogeneity of the film specifically indicating poor protection of the siloxane bonds. It is thus very difficult to obtain reproducible grafting of these molecules. Before fixing or synthesizing oligo-nucleotides to or on the substrate, additional surface reactions are necessary to reduce steric interference at the surface (for example, grafting of bifunctional heterocyclic molecules, as described by L. A. Chrisey et al.), to render the surface more hydrophilic (U. Maskos et al. describe the grafting of ethylene glycol, of pentaethylene glycol or of hexaethylene glycol) and/or to overcome the low reactivity of the surface functional groups, which additional operations are themselves also not controlled.

A. Ulman has described, in Chem. Rev., 1996, 96, 1533-1554, the formation of organized self-assembled mono-layers on solid supports using organosilicon compounds of functionalized alkyltrichlorosilane type. Their use for the fixing of biomolecules is proposed, which method probably necessitates, in this context, a modification of the biomolecule by a thiol functional group and a modification of the surface by hetero-bifunctional molecules.

Organosilicon compounds have been disclosed in the document WO 01/53303 for overcoming these disadvantages. These compounds can be used as coupling molecules in order to deposit an organized self-assembled monolayer at the surface of a solid support. Such molecules have been used in particular for the immobilization of biomolecules, such as nucleic acids.

In contrast to the methods of grafting biomolecules of the prior art, the method disclosed in WO 01/53303 makes it possible to obtain a solid support grafted by biomolecules immobilized on an organized self-assembled monolayer. This characteristic is reflected by the presentation of a homogeneous surface of biomolecules which favors the capture of a minority population of cells having a low density of specific sensors in a biological sample circulating over this surface.

This is because the graftings of the prior art did not make it possible to obtain a satisfactory surface homogeneity and a consequence of this failing is a low level of capture of minority target cells within a biological sample.

In the device of the invention, the solid support is modified by an organized self-assembled monolayer comprising at least one organosilicon compound corresponding to the formula (I):

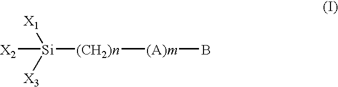

in which:
n is between 15 and 35, preferably between 20 and 25,
m is equal to 0 or to 1,
X₁, X₂ and X₃, which can be identical to or different from one another, are selected from the group consisting of saturated, linear or branched, C₁ to C₆ alkyl groups and hydrolyzable groups, at least one of X1, X₂ or X₃ representing a hydrolyzable group,
A represents an —O—(CH₂—CH₂—O)ₖ—(CH₂)ᵢ— group in which k represents an integer between 0 and 100, preferably between 0 and 5, and i represents an integer of greater than or equal to 0, preferably equal to 0 or to 1,
B represents a group chosen from —OCOR, —OR, —COOR, —R—, —COR, —NR₁R₂, —CONR₁R₂, COOR, —SR or a halogen atom,
R, R₁ and R₂ being chosen from: a hydrogen atom, a saturated or unsaturated and linear or branched hydrocarbon chain which is optionally substituted by one or more halogen atoms and which comprises from 1 to 24 carbon atoms, preferably from 1 to 6 carbon atoms, or an aromatic group optionally substituted by one or more halogen atoms.

The term “aromatic” is understood to mean any group which has one or more aryl rings, for example a phenyl ring.

The term “organized self-assembled monolayer” is understood to mean an assemblage of molecules in which the molecules are organized, which organization is due to interactions and to strong cohesion between the chains of the molecules, giving rise to a stable and ordered anisotropic film (A. Ulman, Chem. Rev., 1996, 96, 1533-1554).

An organized self-assembled monolayer formed on a solid support makes it possible to obtain a dense and homogeneous organic surface with well defined parameters, both chemically and structurally. The formation of this monolayer, obtained by virtue of the self-assembling properties of the compounds of formula (I) for well defined values of n, m, k and i, is perfectly reproducible for each organosilicon compound. In addition, the formation of a very dense organized self-assembled monolayer protects the siloxane bonds with regard to chemical treatments (acidic or basic treatments), which makes it possible to carry out varied chemical reactions on this surface.

The organosilicon compounds of formula (I) used in the present invention advantageously exhibit highly varied functional groups and high reactivity, in view of the nature of the A group and of the diversity of the end B groups which can be used, it being possible, of course, for these B groups to be modified and functionalized as desired according to organic chemistry reactions well known to a person skilled in the art.

The compounds described above make it possible, particularly advantageously and because of the organosilicon compounds of formula (I) selected, to immobilize biomolecules reliably and reproducibly on a support, in view of the homogeneity and the stability. of the organized self-assembled monolayer formed on the support. The biomolecules are immobilized on the modified support via strong covalent bonds, without weakening of the siloxane bonds developed between the organosilicon compounds and the solid support.

Suitable solid supports are generally those having a hydrated surface and/or those having a surface exhibiting hydroxyl groups. Preferably, said support is selected from the group consisting of glasses, ceramics (for example, of oxide type), metals (for example, aluminum or gold) and semimetals (such as silicon).

Within the meaning of the present invention, the term “hydrolyzable” is understood to mean any group capable of reacting with an acid in an aqueous medium so as to give the compounds X₁H, X₂H or X₃H, X₁, X₂ and X₃ being as defined in the formula (I).

According to an advantageous embodiment, said hydrolyzable group is selected from the group consisting of halogen atoms, the —N(R′)₂ group and —OR′ groups, R′ being a saturated, linear or branched, C₁ to C₆ alkyl group.

As regards the B groups and the hydrolyzable groups, suitable halogen atoms are equally well fluorine as chlorine, bromine or iodine.

According to another advantageous embodiment, X₁, X₂ and X₃ represent chlorine atoms.

Advantageously, n is greater than or equal to 22.

The use of modified solid supports according to the present invention is particularly advantageous in the preparation of supports to which are covalently fixed protein entities (antibody, ligand for a cell receptor, protein, and the like) capable of selectively fixing cells representing a subpopulation within a biological sample.

The preparation of the grafted solid support according 10 to the present invention comprises the following stages:
a) preparation of a solid support modified by an organized self-assembled monolayer comprising at least one organosilicon compound corresponding to the formula (I) as defined above, in which said organosilicon compounds exhibit, at their end, a protected carboxylic acid, hydroxyl or amine functional group;
b) optionally deprotection of the carboxylic acid, hydroxyl or amine functional group;
c) optionally grafting of a coupling agent to the modified solid support;
d) grafting of the protein entity.

Stage a) is advantageously carried out via the following stages:
i) removal of the contaminants from the solid support and hydration and/or hydroxylation of its surface,
j) introduction, into a mixture of at least two solvents comprising at least one nonpolar hydrocarbon solvent, under an inert atmosphere, of an organosilicon compound of formula (I) as defined above, said compound exhibiting, at one end, a protected carboxylic acid, hydroxyl or amine functional group,
k) silanization of the support obtained in stage i) by immersion in the solution prepared in stage j),
l) optionally, annealing of the silanized support obtained in stage k), carrying the self-assembled monolayer, at a temperature of between 50 and 120° C., for a period of time from 5 minutes to overnight, and
m) rinsing the modified support obtained in stage k) or l) with the acid of a solvent, preferably a polar solvent.

The preparation of solid supports functionalized by a layer of silanes is illustrated in detail in the document WO 01/53303.

The term “contaminants” of the solid support is understood to mean any compound, such as grease, dust or others, present at the surface of the support which does not form part of the chemical structure of the support itself.

According to the nature of the solid support, stage i) can be carried out using one or more solvents and/or oxidizing agents and/or hydroxylating agents (for example, a chromic acid/sulfuric acid mixture), a solution of detergent (for example Hellmanex®), a photochemical treatment with ozone or any other appropriate treatment.

Stage j) can advantageously be carried out in a mixture of at least one nonpolar hydrocarbon solvent and of at least one polar solvent. In this case, the proportions by volume of nonpolar solvent and of polar solvent are preferably between 70/30 and 95/5.

By way of example and without implied limitation, during stage j), a nonpolar hydrocarbon solvent which can be used is cyclohexane and a polar solvent which can be used is chloroform.

During stage j), the concentration of the organosilicon compound in the mixture of solvents is preferably between 1×10⁻⁵ and 1×10⁻² mol/liter.

Stage k) of silanization of the support can be carried out for a time of between 1 minute and 3 days and at a temperature of between −10° C. and 120° C., according to the solvents used.

Stage c) of grafting a coupling agent can be carried out in a different way depending on the nature of the end functional group of the group of formula (I).

(i) In the case of the chains comprising an —NH₂ ending:

A homobifunctional or heterobifunctional coupling agent can be chosen. The functional group intended to react with the protein is either the carbonyl in has of the sulfosuccinimidyl (BS3) or the sulfo-NHS ester (Sulfo-SMCC). The following may be involved:
BS3: bis(sulfosuccinimidyl) suberate (Staros J V (1982). N-hydroxysulfosuccinimide active esters: Bis(N-hydroxysulfosuccinimide) esters of two dicarboxylic acids are hydrophilic, membrane-impermeant, protein cross-linker. Biochemistry, 21, 3950), which is employed according to scheme 1:

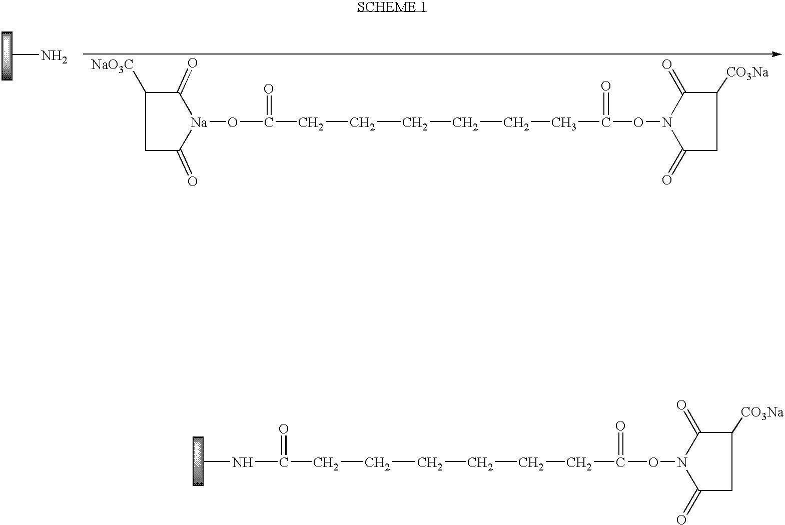

Sulfo-SMCC: sulfosuccinimidyl 4-(N-maleimido-methyl)-1-cyclohexanecarboxylate (Samoszuk M K et al. (1989). Antibody, Immunoconjugates. Radiopharm., 2, 37), which is employed according to scheme 2:

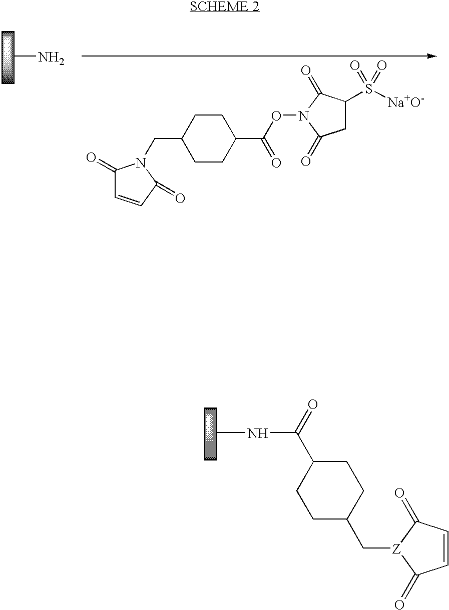

Instead of a chemical coupling agent, protein A (PA) or protein G (PG) can act as coupling agent (Eliasson M, Olsson A, Palmcrantz E et al. (1988), Chimeric-binding receptors engineered from Staphylococcal protein A and Streptococcal protein G. J. Biol. Chem., 263, 4323). In this case, PA or PG can be coupled directly to the NH₂-terminated chlorosilane chain in the presence of EDC [1-ethyl-3-(3-dimethyl-aminopropyl)carbodiimide hydrochloride] (Grabarek Z and Gergely J (1990). Zero-length crosslinking procedure with the use of active esters. Anal. Biochem., 185, 131). This alternative form is illustrated by scheme 3:

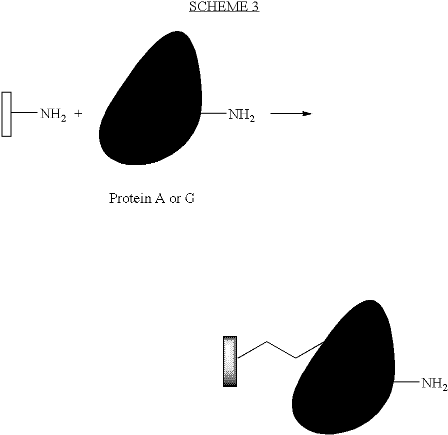

(ii) In the case of the OH-terminated chains:

A heterobifunctional coupling agent is:
PMPI: N-(p-maleimidophenyl) isocyanate (Annunziato ME at al. (1993). p-Maleimidophenyl isocyanate: a novel heterobifunctional linker for hydroxyl to thiol coupling. Bioconjug. Chem., 4, 212), which is used as illustrated by scheme 4:

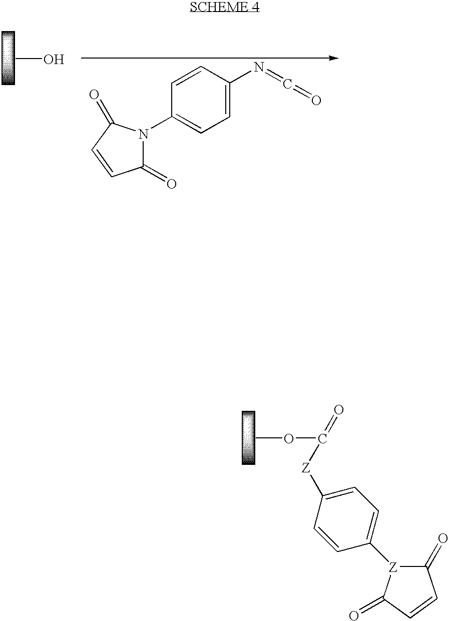

(iii) In the case of the COOH-terminated chains:

The heterobifunctional coupling agent is:
KMUH: κ-maleimidoundecanoic acid N-hydrazide (Trail PA et al. (1993). Science, 261, 212), which is used as illustrated by scheme 5:

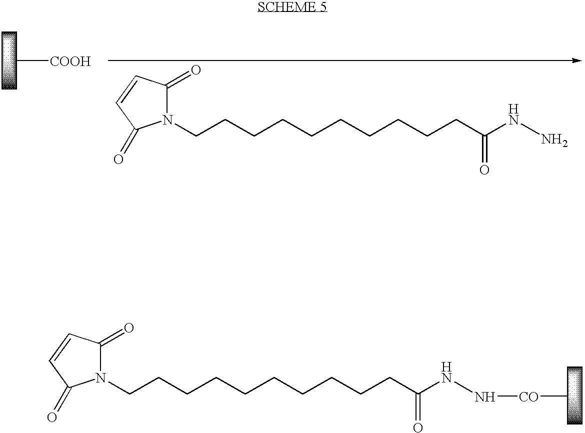

In the case of the chains comprising an —COOH ending, it is also possible to use protein A or protein G, as described above.

In stage d), the protein entity is chosen according to the use which it is desired to make of the device:

The nature of the subpopulation of cells which it is desired to capture is determining for the choice of this protein entity. The latter has to exhibit a specific affinity for the targeted subpopulation.

The strategy for the capture of the CTCs is based on the recognition properties of the molecules of the cell surface. These molecules are cell adhesion molecules or membrane receptors. Cell capture is mediated by the interaction of specific ligands with its receptors or of antibodies directed against an epitope of an adhesion molecule or of a receptor.

Whatever the protein entity used for cell capture (ligand or antibody), it is necessary to modify the side NH₂ functional groups of the lysines of the recognition protein and to convert them to thiol (SH) groups.

In the case of an antibody, for antigen-antibody recognition, the modification of the side NH₂ of the lysine situated on the Fc portion is of great importance as it makes it possible to leave the binding sites free on the Fab fragments, these sites being necessary for the recognition of the cell antigens.

The strategy for fixing the protein recognition entity can consist of the use of the reactant SATA (N-Succinimidyl S-AcetylThioAcetate) by a process comprising two stages (Duncan R J S, Weston PD et al. (1983). A new reagent which may be used to introduce sulfhydryl groups into proteins, and its use in the preparation of conjugates for immunoassays. Anal. Biochem., 132, 68), as illustrated in schemes 6 and 7, or of Traut's reagent (2-iminothiolane.HCl), as illustrated in scheme 8 (Ghosh S S, Kao P M, McCue A W et al. (1990). Use of maleimide thiol coupling chemistry for efficient syntheses of oligonucleotide-enzyme conjugate hybridization probes. Bioconjug. Chem., 1, 71):

A. Reaction of the NH₂ Groups with SATA (Stage I)

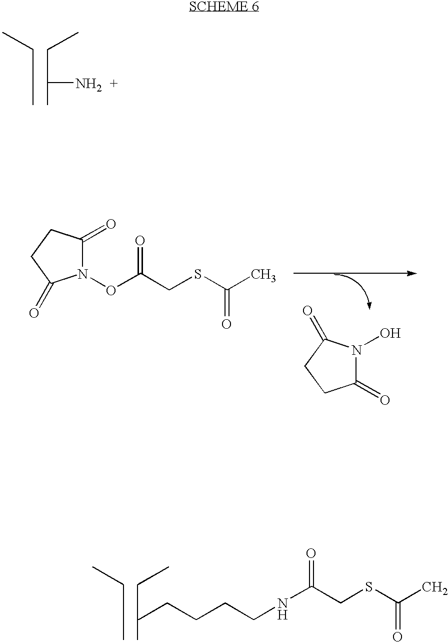

B. Deprotection of the SH Groups by Hydroxylamine (Stage II)

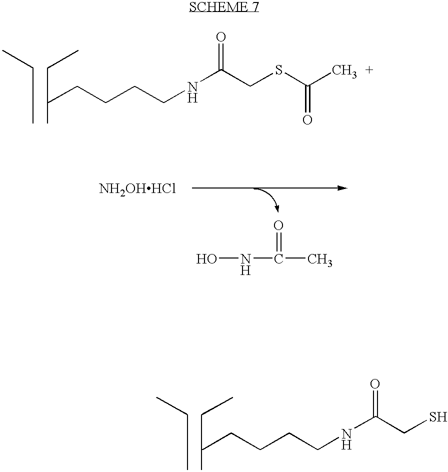

C. Modification of the NH₂ Groups by Traut's Reagent

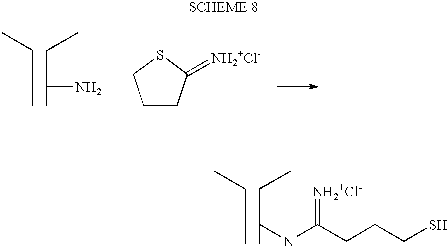

A few examples of antibodies which recognize surface molecules and which are intended to be grafted in the device of the invention:

a) Overexpression of the Intercellular Adhesion Molecule Ep-CAM

Solid tumors of the breast deriving from epithelial tissues overexpress Ep-CAM, an intercellular adhesion molecule (Gastl G, Spizzo G. Obrist P et al. (2000). Ep-CAM overexpression in breast cancer as a predictor of survival. The Lancet, 356, 1981). Ep-CAM is also denoted under the names: 17-1A, ESA, EGP40. It is a 40 kDa transmembrane epithelial glycoprotein coded by the GA733-2 gene (Linnenbach A J, Woicierowski J, Wu S et al. (1989). Sequence investigation for the major gastrointestinal tumor-associated antigen family, GA733. Proc. Natl. Acad. USA, 86, 27; Gottlinger H G, Funke I, Johnson J P et al. (1986). The epithelial cell surface antigen, a target for antibody-mediated tumor therapy: its biochemical nature, tissue distribution and recognition by different monoclonal antibodies. Int. J. Cancer, 38, 47). It is recognized as a tumor antigen overexpressed in the majority of carcinomas and is involved in the metastatic process (Litvinov S V, Velders M P, Bakker H A et al. (1994). Ep-CAM: a human antigen is a homophilic cell-cell adhesion molecule. J. Cell Biol., 125, 437). Ep-CAM is suggested as therapeutic target, in particular in immunotherapy. Various monoclonal antibodies, 17-1A, MOC31, Ber-EP4 and HA-125, are directed against various epitopes of this molecule. The monoclonal antibody Ber-EP4 recognizes two 34 and 39 kDa epitopes of Ep-CAM (Latza U, Niedobitek G, Schawarting R et al. (1990). Ber-EP4: a new monoclonal antibody which distinguishes epithelia from mesothelia. J. Clin. Pathol., 43, 213).

Ber-EP4 or MOC31 or HA-125 can be bonded either directly to protein A or protein G or to the chlorosilane chain comprising an NH₂, OH or COOH ending via an appropriate coupling agent.

b) Overexpression of HER-2

The gene of the human growth factor 2 receptor (HER-2)/neu (c-erbB-2) is located on the 17q chromosome and codes for a transmembrane protein receptor with a tyrosine kinase activity of the family of the epidermal growth factor receptors (EGFRs) or the family of the HERs. The amplification of the HER-2/neu gene or the overexpression of the HER-2 (p185^{HER2}) protein are prognosis factors in breast cancer and various carcinomas (Slamon D J, Clark G M, Wong S G et al. (1987). Human breast cancer: correlation of relapse and survival with amplification of the Her-2/neu oncogene. Science, 235, 177). The anti-HER-2 antibody (trastuzumab) is used in antitumor therapy.

The anti-HER-2 (anti-human ErB2) antibody is used as specific marker for capturing the CTCs of breast cancer. In association with the detection by the Ber-EP4 antibody, it will act as potential marker for the CTCs.

The grafting of the anti-HER-2 antibody can be carried out in the same way as that of the Ber-EP4 antibody.

c) Overexpression of MUC1

Mucins are expressed by various types of normal epithelial cells in a rough environmental context, such as the air/water interface of the respiratory system, the acid environment of the stomach, the complex environment of the intestines and the secretory epithelial surfaces of specialized organs, such as the liver, pancreas, gall bladder, kidneys, salivary glands, lacrymal glands and eye. The family of the mucins comprises at least 17 secreted or nonsecreted molecules and plays a central role in the maintenance of homeostatis (Hollingsworth M A and Swanson J B (2004). Mucins in cancer: protection and control of the cell surface. Nat. Reviews Cancer, 4, 45).

Mucins are high molecular weight glycoproteins with repetitions in tandem of serine-, threonine- and proline-rich sequences attached to oligosaccharides via O-glycoside bonds. MUC1 is an integral membrane protein. In breast tumors, MUC1 is overexpressed and glycosylated aberrantly (Rahn J J, Dabbagh L, Pasdar M et al. (2001). The importance of MUC1 cellular localization in patients with breast carcinoma: an immunohistologic study of 71 patients and review of the literature. Cancer, 91, 1973).

The anti-MUC1 (CT2 Mab) antibody is used as specific marker for capturing the CTCs of breast cancer. In association with the detection by the Ber-EP4 antibody, it will act as a potential marker for the CTCs.

The grafting of the anti-MUC1 antibody can be carried out in the same way as that of the Ber-EP4 antibody.

The membrane antigens chosen comprising a membrane glycoprotein Ep-CAM, the glycoprotein MUC1 and a growth factor receptor HER-2/neu are tumor markers over-expressed in the CTCs. For this reason, the tumor cells may display an optimized density of cell surface molecules, thus favoring specific cell capture. It should be noted that the density of CTC in the peripheral blood is a completely unknown parameter. This parameter can vary according to the extent of the pathology and is about 1 CTC per 10³ to 2×10⁴ leukocytes.

The antibodies directed against these molecules are specific and sufficiently selective to make possible CTC capture under the best possible conditions in terms of preservation, of cell morphology and of viability.

The strategy of using long organosilicon chains (n=22) for the grafting of the surfaces, the addition of a coupling agent and the grafting of the Fc portion of the antibody are assets in optimizing cell capture.

This is because the length of the X₃—Si—(CH₂)₂₂-coupling agent-antibody combination varies between 40 and 50 Å, thus producing great flexibility in a favorable presentation of the antibody to the cell antigen.

This is because, allowing a density of overexpressed HER-2 receptors on the overall surface of the tumor cell of 10⁶, a rapid evaluation gives 8 receptors in a square area with a length per side of 10 Å. According to the evaluation by atomic force microscopy (AFM), the grafting density of organosilicon chains being from 3 to 5 molecules per nm², this configuration is highly favorable to good grafting of bonding agent and of antibody being obtained.

### EXAMPLE

1—Device

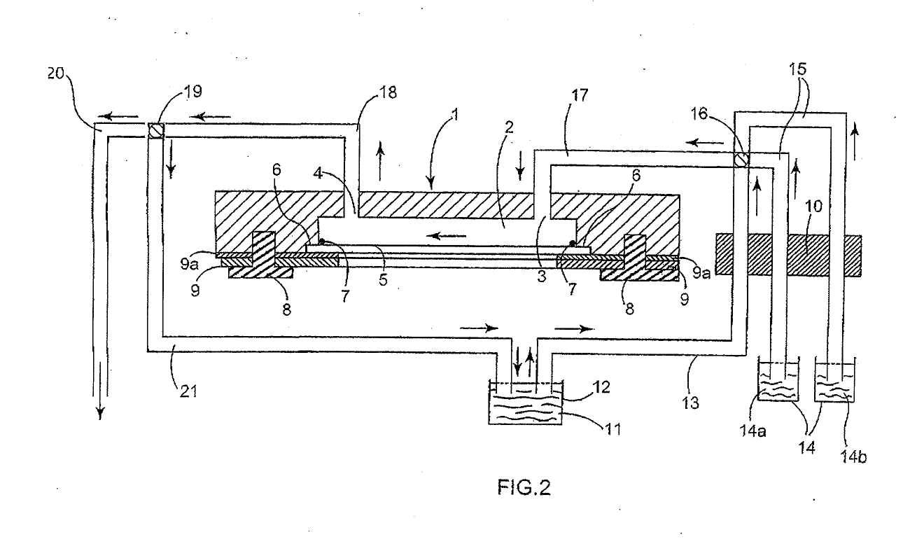

Cell capture by means of a laminar flow chamber is illustrated in FIG. 2.

The present cell capture process uses a laminar flow chamber (1), manufactured from Plexiglas®, comprising a cavity (2) with dimensions of 20×6×0.2 mm³ (l×w×h) which is connected to the outside via inlet (3) and outlet (4) openings for the circulation of the cells and fluids.

The wall (5) constituting the floor of the chamber is a removable glass or crystalline Si slide which is chemically functionalized. This slide is applied in a recess (6) against the cavity (2) of the chamber (1) by means of an O-ring (7) intended to provide for the leaktightness thereof. Provision may be made for the ends of the cavity (2) to be rounded so as to favor the positioning of the O-ring (7). The slide is screwed (8) to the Plexiglas® base via a metal plate (9). A Teflon® seal (9a) separates the glass slide from the metal plate. Any other means known to a person skilled in the art which makes possible the fixing of the solid support in the cavity can be used. In the case illustrated in FIG. 2 and according to a preferred alternative form of the invention, the metal plate (9) comprises, in its central part, a transparent region (not shaded) which makes it possible to observe the interior of the cavity (2) using a microscope (not represented). Likewise for the Teflon® seal (9a) comprises a transparent central region (not shaded). The proportion of the opaque and transparent regions can vary.

The deliveries and discharges of the fluids via the openings (3, 4) are placed under the control of a peristaltic pump (10) which sets the throughput and flow rate.

The peristaltic pump (10) withdraws the biological sample (11) from a receptacle (12), provided for this purpose, closed so as to be kept sterile, and injects it into the circulation tube (13). It can withdraw reagents (14a, 14b) from the receptacles (14), also closed so as to provide for the good preservation of the products which they comprise, and inject them into the circulation tubes (15). A valve (16) at the intersection of the circulation tubes (15), (13) and (17) makes it possible to regulate the connection between the circulation tubes (15) and (13) and the tube (17) for introduction into the laminar flow chamber (1). The fluids pass through the laminar flow chamber (1) under the control of the peristaltic pump (10) and exit therefrom via the outlet tube (18). A valve (19) makes it possible to direct the fluid from the outlet tube (18) either to a discharge tube (20) or to a recycling tube (21) connected to the cavity (2) which makes it possible to again pass the fluid through the laminar flow chamber (1). In FIG. 2, the recycling tube (21) is connected to the cavity (2) via the receptacle (12) but it is possible to provide for a direct connection between the recycling tube (21) and the cavity (2). The recycling of the biological sample via this circuit makes possible better effectiveness in capturing the target cells. The direction of the circulation of the fluids in the device is shown by arrows.

The device described above constitutes an implementational example of the invention. Other alternative forms are included within the scope of the invention. The essential characteristic lies in circulating the biological sample through a chamber for circulation of fluids which makes possible flow under laminar conditions, an internal wall of which is provided with specific grafting described above. The circuit for circulation of fluids, apart from the chamber for circulation of fluids, can be adjusted according to the reagents which it is or is not desired to use. According to an alternative form of the invention, the device can comprise several chambers for circulation of fluids placed in series or in parallel. This chamber for circulation of fluids can be included in any microfluidic device with a configuration appropriate for the capture of cells, such as, for example, a microfluidic device such as that disclosed in the document U.S. Pat. No. 6,408,878.

When the biological sample has passed through the chamber for circulation of fluids one or more times, depending on the experimental protocol chosen, it is possible to recover the cells which have become fixed to the grafted wall according to the invention by injecting, into the chamber for circulation of fluids, a reagent which makes possible the detaching of these cells. They are subsequently recovered with a view to being analyzed and counted.

According to an alternative form of the invention, the device for FIG. 2 can be constructed in the following way:

A solid support grafted by chlorosilane functional groups comprising a protected hydroxyl ending, a protected amine ending or a protected acid ending is fixed in the cavity (2) as described above, so as to close the cavity (2). The other mechanical components of the device are placed as in FIG. 2. A reagent for deprotecting the end functional groups is injected into the cavity (2) from one of the receptacles (14), then a coupling agent is injected into the cavity (2) and, finally, the recognition biomolecule. This is because it is possible, from the mechanical device described in FIG. 2, to functionalize the wall (solid support) grafted by the chlorosilanes by an appropriate sequence of injections of reagents, it being possible for rinsing stages to be provided as intermediate stages.

2—Protocol for Isolation of the CTCs

A volume of 3 to 5 ml of peripheral blood from patients suffering from the spread of breast cancer and/or from cancer-free controls is withdrawn conventionally into a Hanks' medium (In VitroGen) comprising 5 mM of EDTA (ethylenediaminetetraacetic acid). The nucleated cells (leukocytes and tumor cells) are subsequently separated from the red blood cells and platelets over a gradient comprising Ficoll (Amersham) in tubes rendered appropriate beforehand (Leucosep). The cell fractions comprising the leukocytes and the CTCs are subsequently diluted in Hanks' medium comprising 0.1% of HSA (human serum albumin) in a proportion of 1×10⁶ cells per ml before isolating the cells by means of the laminar flow chamber. The throughput of the peristaltic pump is adjusted to approximately 50 μl/min, corresponding to a shear rate of 15 s⁻¹ and a shear stress of 0.15 dync/cm².

The CTCs isolated can be recovered by dissociation of the antigen-antibody bond by various methods: by competition, using a peptide inhibitor; or by increasing the rate of flow by a factor of 10 to 100, or by dissociation of the S=S bridges of the fragments of the antibodies for the release of the captured cells.

Other tests were carried out on microfluidic devices and laminar flow chambers, the dimensions of which are given in tables 1 and 2 below:

TABLE 1
Dimensions of the laminar flow chamber

| l × w × h (mm³) | Volume (μl) |
|---|---|
| 20 × 6 × 0.10 | 12 |
| 20 × 6 × 0.15 | 18 |
| 20 × 6 × 0.20 | 24 |
| 20 × 6 × 0.25 | 30 |

TABLE 2
Dimensions of the microfluidic device

| l × w × h (μm³) | Volume (nl) |
|---|---|
| 100 × 50 × 200 | 1 |
| 100 × 100 × 200 | 2 |
| 150 × 100 × 200 | 3 |
| 150 × 150 × 200 | 4.5 |

3—Molecular Characterization of the Tumor Phenotype

Various processes for the characterization of the isolated CTCs can be applied.

a) Morphological, Immunocytological and Genetic Characterization of the CTCs
by immunohistochemistry (IHC): detection of marking by antibodies: antikeratin (CK19, CK20, CK22), anti-CD45 specific for leukocytes
Tumor markers (XHC and RT-PCR): overexpression of HER-2/neu, telomerase, MUC-1

b) Cytogenetic Characterization
CGH (comparative in situ hybridization)
FISH (fluorescence in situ hybridization)

4—Other Applications

a) Prenatal Diagnosis

The present process can also be used in obstetrics in prenatal diagnosis for early genetic analyses of fetal cells in maternal blood (Bianchi D. (1999). Fetal cells in the maternal circulation: feasibility for prenatal diagnosis. Br. J. Maematol., 105, 574; Fisk N. (1998). Maternal-fetal medicine and prenatal diagnosis. Curr. Opin. Obstet. Gynecol., 10, 81). The population of fetal cells targeted in our process is composed of trophoblast cells of epithelial type and with a short lifetime during the first trimester of gestation (Shulman L P. (2003). Fetal cells in maternal blood. Current Women's Health Reports 3, 47). The density of this population of cells is very low: 1 fetal tropho-blast per 10⁶ maternal cells. Cell capture can be carried out using antibodies directed against epithelial membrane antigens or an antibody directed against human placental lactogen (Latham S E, Suskin H A, Petropoulos A et al. (1996). A monoclonal antibody to human placental lactogen hormone facilitates isolation of fetal cells from maternal blood in a model system. Prenat. Diagn., 16, 813).

The present process exhibits a certain advantage with respect to the existing methods, such as flow cytometry or magnetic beads.

The fetal cells isolated are subjected to molecular genetic characterization for the detection of genetic abnormalities (PCR, FISH).

b. Detection of CTCs in Peripheral Blood and Bone Marrow

Another application of the present process relates to the detection of CTCs in bone marrow and peripheral blood (Chapter 15: Tumor Cell Contamination (2001). Autologous Blood and Marrow Transplantation X: Proceedings of the Tenth International Symposium, edited by Karel A. Dicke and A. Keating).

In the first case, the presence of metastatic CTC in bone marrow makes it possible to evaluate the prognosis in order to target the therapies.

In the second case, the present process relates to a two-stage evaluation on the same patient: 1) with regard to the presence and the percentage of CTC in the peripheral blood before marrow ablative chemotherapy and 2) the absence of CTC with regard to the blood sample treated and purged in order to obtain a population of hematopoietic strain cells for an autologous transplantation.

1. A microfluidic device for the capture of a population of cells comprising a chamber for circulation of fluids the dimensions of which are chosen so that it is a laminar flow chamber and an internal wall of which is provided with grafting by an organized self-assembled layer of silane to which is fixed a layer of recognition biomolecules specific for the population of cells, characterized in that the organized self-assembled layer of silane comprises at least one organosilicon compound corresponding to the formula (I): 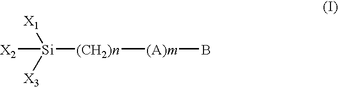
in which:
n is between 15 and 35, preferably between 20 and 25,
m is equal to 0 or to 1,
X₁, X₂ and X₃, which can be identical to or different from one another, are selected from the group consisting of saturated, linear or branched, C₁ to C₆ alkyl groups and hydrolyzable groups, at least one of X₁, X₂ or X₃ representing a hydrolyzable group,
A represents an —O—(CH₂—CH₂—O)ₖ—(CH₂₎ᵢ— group in which k represents an integer between 0 and 100, preferably between 0 and 5, and i represents an integer of greater than or equal to 0, preferably equal to 0 or to 1,
B represents a group chosen from —OCOR, —OR, —COOR, —R—, —COR, —NR₁R₂, —CONR₁R₂, COOR, —SR or a halogen atom,
R, R₁ and R₂ being chosen from: a hydrogen atom, a saturated or unsaturated and linear or branched hydrocarbon chain which is optionally substituted by one or more halogen atoms and which comprises from 1 to 24 carbon atoms or an aromatic group optionally substituted by one or more halogen atoms, it being understood that, when R═H or R=alkyl, then B≠—R.

2. The device as claimed in claim 1, wherein the layer of biomolecules is bonded to the organized self-assembled layer of silane via a layer of coupling agent.

3. The device as claimed in claim 1, wherein the internal wall f the chamber for circulation of fluids provided with specific grafting is composed of a solid support which can be a glass or silicon slide or any solid metal surface comprising —OH functional groups at the surface.

4. The device as claimed in claim 1, wherein the chamber for circulation of fluids comprises a cavity of parallelepipedal type comprising two orifices placed at two ends of the cavity, the fluid being injected via a first orifice and collected via a second orifice.

5. The device as claimed in claim 1, wherein the chamber for circulation of fluids comprises a cavity with a volume of less than or equal to 30 μl, more preferably still of less than or equal to 12 μl.

6. The device as claimed in claim 1, additionally including a pump which makes it possible to inject the sample fluid into the chamber for circulation of fluids.

7. The device as claimed in claim 1, wherein the wall provided with specific grafting is parallel to the flow which passes through the chamber for circulation of fluids.

8. The device as claimed in claim 1, wherein the hydrolyzable group is selected from: halogen atoms, the —N(R′)₂ group and —OR′ groups, R′ being a saturated, linear or branched, C₁ to C₆ alkyl group.

9. The device as claimed in claim 1, wherein X₁, X₂ and X₃ represent chlorine atoms.

10. The device as claimed in claim 1, wherein n is greater than or equal to 22.

11. The device as claimed in claim 2, wherein the coupling agent is selected from:
bis(sulfosuccinimidyl) suberate;
sulfosuccinimidyl 4-(N-maleimidomethyl)-1-cyclohexanecarboxylate
protein A
protein G
N-(p-maleimidophenyl) isocyanate
K-maleimidoundecanoic acid N-hydrazide.

12. The device as claimed in claim 1, wherein the recognition biomolecule is a protein, the side NH₂ functional groups of the lysines of which have been converted to thiol (SH) groups.

13. The device as claimed in claim 1, wherein the recognition biomolecule is selected from:
monoclonal antibodies 17-1A, MOC31, Ber-EP4 and HA-125, which recognize the intercellular adhesion molecule Ep-CAM,
the anti-HER-2 (anti-human ErB2) antibody
the anti-MUC1 (CT2 Mab) antibody.

14. The device as claimed in claim 1, wherein said chamber comprises a laminar flow chamber including a cavity and inlet and outlet openings for the circulation of cells and fluids, a removable glass or crystalline Si slide which is chemically functionalized and which constitutes the floor of the chamber fixed to the cavity, a peristaltic pump which controls the delivery and the discharges of the fluids via the openings, at least one receptacle in which the biological sample is placed, at least one circulation tube which connects the receptacle to the opening and at least one outlet tube connected to the opening.

15. The device as claimed in claim 14, wherein the receptacle is connected to the opening via a tube for introduction into the laminar flow chamber.

16. The device as claimed in claim 15, comprising at least one receptacle comprising a reagent, a circulation tube which connects the receptacle to the opening via the tube, and a valve at the intersection of the circulation tubes for regulating the connection between the tubes.

17. The device as claimed in claim 14, comprising a discharge tube and a recycling tube connected to the cavity and a valve for directing the fluid between the tubes.

18. A process for capturing cells in a biological sample, said process comprising a stage of passing the biological sample through the chamber for circulation of fluids of a device as claimed in claim 1.

19. The process as claimed in claim 18, comprising at least two stages of passing the biological sample through the chamber for circulation of fluids.

20. The process as claimed in claim 18 comprising an additional stage of detaching the cells.

21. A method for capturing a population of cells in a biological sample comprising circulating the sample in a device as claimed in claim 1.

22. The method as claimed in claim 21, wherein the specific surface markers for which cells have an interaction with their receptors with a strength ranging from 10 pN to 1 nN.

23. A method for the purification and characterization of circulating tumor cells from a blood sample comprising circulating the blood sample in a device as claimed in claim 1.

24. The method as claimed in claim 23 comprising the diagnosis and monitoring of the development of a pathology selected from breast cancer, prostate cancer, kidney cancer, bladder cancer, liver cancer, colon cancer or lung cancer.

25. A method for the purification and characterization of fetal cells from a blood sample from the mother comprising circulating the blood sample in a device as claimed in claim 1.

26. A method for the detection of circulating tumor cells in bone marrow comprising circulating the cells in a device as claimed in claim 1.
